(19) United States
(12) Patent Application Publication
(10) Pub. No.: US 2006/0040275 A1
(43) Pub. Date: Feb. 23, 2006

(54) C-shaped probe

(76) Inventors: David Rosmarin, New York, NY (US); Zhiheng Pei, New York, NY (US); Martin Jack Blaser, New York, NY (US); Sanjay Tyagi, Newark, NJ (US)

Correspondence Address: Andrew M. Parial;Kohn & Associates, PLLC, Suite 410, 30500 Northwestern Highway, Farmington Hills, MI, 48334, US

(21) Appl. No.: 10/966,321

(22) Filed: Oct. 15, 2004

Related U.S. Application Data
(60) Provisional application No. 60/512,001, filed on Oct. 16, 2003.

(51) Int. Cl. C12Q 1/68; C07H 21/04

(52) U.S. Cl. 435/6; 536/24.1

(57) ABSTRACT

A nucleic acid probe for identifying a target nucleic acid sequence including a linker structure including a 5′ end and a 3′ end; 5′ marker mechanism for producing an identifying signal to the target nucleic acid sequence, wherein the 5′ marker mechanism is conjugated to the 5′ end of the linker structure; and 3′ marker mechanism for producing an identifying signal to a target nucleic acid sequence, wherein the 3′ marker mechanism is conjugated to the 3′ end of the linker structure and wherein identification of the target nucleic acid sequence occurs when the 5′ marker mechanism and 3′ marker mechanism are in close physical proximity to each other. A nucleic acid probe for identifying a target nucleic acid including a marker pair mechanism for producing an identification signal to identify a target nucleic acid sequence; and linker mechanism for linking the marker pair mechanism together. A biosensor including a fluorophore quencher pair; and a linker sequence for linking the fluorophore quencher pair. A probe for use in a polymerase chain reaction (PCR).

### CROSS REFERENCE TO RELATED APPLICATIONS

This application claims the benefit of priority under 35 U.S.C. Section 119(e) of U.S. Provisional Patent Application No. 60/512,001, filed Oct. 16, 2003, which is incorporated herein by reference in its entirety.

### BACKGROUND OF THE INVENTION

1. Technical Field

The present invention relates to the field of genetics and more specifically towards nucleic acid probes for use in nucleic acid analysis, detection, and sequencing.

2. Background Art

Nucleic acids form the genetic material of all living organisms. Nucleic acids are involved in the control and regulation of gene expression. Nucleic acids encode polypeptides such as enzymes, structural proteins, and other effectors of biological functions. Thus, by studying the presence or absence of various nucleic acid sequences, various cell functions can be determined.

Nucleic acids have the ability to form sequence-specific hydrogen bonds (i.e., hybridize) with other nucleic acids having a complementary nucleotide sequence. A nucleic acid sequence having known nucleotides can be used as a probe in a test sample to hybridize with a “target” nucleic acid sequence having a complementary nucleic acid sequence. Then, by labeling the probe with a marker, the presence or absence of the “target” nucleic acid sequence can be determined. Because all strains of a particular organism or virally-infected cell share a genetic component in the form of nucleic acids, hybridization assays are valuable research and diagnostic tools for detection of and diagnosis of various disease states in humans, animals, and plants. Additionally, the ability to probe for a specific nucleotide sequence is of potential value in the identification and diagnosis of human genetic disorders.

Probes have become of great importance in the clinical setting, in testing CSF (Kristiansen, B. et al., Jaton, K., et al., Greisen, K., et al., Backman, A., et al. and Schurrman, T., et al.) testing synovial fluid (Stuhlmeier, R., et al., Cox, C J, et al., and Jalava, J., et al.), testing blood (van Haeften, R., et al., and Wellinghausen, N., et al.), and testing in other sterile sites for bacteria. There is also a potential role in genetic testing for mismatches in the human genome. Recently, probes have also been used to follow RNA within a cell. However, improvements are needed to increase sensitivity, increase (or decrease) specificity, ease synthesizing, decrease cost, decrease the false background in “dirty” systems such as cells, or have a stable signal in the presence of excess target.

Numerous probes exist in the prior art. There are many probe designs such as the Q-beta replicase probe, molecular beacons, and padlock probe among others. One of the most widespread designs used, due to its low cost and simplicity, is two binary probes.

The various probes of the prior art have numerous drawbacks. For example, some prior art probes do not have great sensitivity because of a greater loss of entropy due to the use of separate marker and complementary probe sets. Additionally, other prior art probes require lengthy complementary nucleic acid sequences to hybridize with a target nucleic acid sequence. Moreover, the probes of the prior art require additional steps and washes and cannot be used in homogenous solutions. For probes that cannot be used in homogenous solution “it is necessary to label the oligonucelotide probes, immobilize the hybrids on a solid surface, remove the unhybridized probes, and then determine the number of probes that remain. The requirement that unhybridized probes be removed precludes the use of hybridization for real-time monitoring of nucleic acid synthesis and for locating specific nucleic acids in living cells.” (S. Tyagi, et al.). Specifically with regard to binary probes, their problems include: 1) decreased signal if excess target in binary probes; 2) binary probes have to be matched in hybridization temperature; 3) binary probes require long stretches of target; and 4) a specificity based on small hybridization temperature differences exists.

Accordingly, there is a need for alternative probes that detect specific nucleic acid sequences, provide increased sensitivity and specificity, and avoid the deficiencies of the other methods and systems of the prior art.

### SUMMARY OF THE INVENTION

The present invention provides a nucleic acid probe for identifying a target nucleic acid sequence including a linker structure including a 5′ end and a 3′ end; 5′ marker mechanism for producing an identifying signal to a target nucleic acid sequence, wherein the 5′ marker mechanism is conjugated to the 5′ end of the linker structure; and 3′ marker mechanism for producing an identifying signal to the target nucleic acid sequence, wherein the 3′ marker mechanism is conjugated to the 3′ end of the linker structure and wherein identification of the target nucleic acid sequence occurs when the 5′ marker mechanism and 3′ marker mechanism are in close physical proximity to each other. Further provided is a nucleic acid probe for identifying a target nucleic acid sequence including a marker pair mechanism for producing an identification signal to identify a target nucleic acid sequence; and linker mechanism for linking the marker pair mechanism together. In addition, the present invention provides a biosensor including a fluorophore quencher pair; and a linker sequence for linking the fluorophore quencher pair. The present invention also provides a probe for use in rapid polymerase chain reaction (PCR).

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1A illustrates one embodiment of the nucleic acid probe of the present invention, FIG. 1B illustrates an example of a binary probe of the prior art, FIG. 1C illustrates an example of a K-Probe of prior art, and FIG. 1D illustrates an example of a probe of the prior art;

FIG. 2 is a plot of the fluorescence of the control light cycler probes as a function of temperature in a series of different concentrations of target DNA present;

FIG. 3 is a plot of the fluorescence of the C-shaped probe as a function of temperature in a series of different concentrations of target DNA present, wherein as the temperature goes down, hybridization of the probe to the target nucleic acid sequence occurs, and the fluorescent donor is fixed into a conformation immediately next to a fluorescent quencher on the end of the probe;

FIG. 4 is a plot that illustrates that as the C-shaped probe arm length increases, hybridization of the probe to the target DNA can be achieved at increasingly higher temperatures (for monitoring products in PCR, it is necessary to detect the target at temperatures above 50° C.);

FIG. 5 is a plot that illustrates that as the C-shaped probe arm length is increased to a length of 14 or 16 nucleotides, an increased fluorescence signal is achieved (as well as hybridization at higher temperatures), wherein “arm 6” represents a C-shaped probe arm length of 6 nucleotides as part of each arm;

FIG. 6 illustrates various embodiments of the present invention and the advantages of the present invention over the prior art, wherein FIG. 6A illustrates one embodiment of the present invention, FIG. 6B illustrates another embodiment of the present invention demonstrating that the probe can include arms with differing base pairs, FIG. 6C illustrates that the probe of the present invention can be simultaneously sensitive and specific, and FIG. 6D illustrates the binding of the probe of the present invention to a double stranded DNA sequence;

FIG. 7 is a hybridization curve comparing the C-shaped probe of the present invention with a binary pair control, wherein the hybridization curve demonstrates that the C-shaped probe generates a stronger signal and hybridizes faster than the binary pair controls;

FIG. 8 is a chart demonstrating that the C-shaped probe of the present invention can quantitatively measure the concentration of target nucleic acid sequence, wherein in the range of excess probe, the signal varies approximately linearly with target concentration, and the excess target does not appreciably decrease the signal of the C-shaped probe;

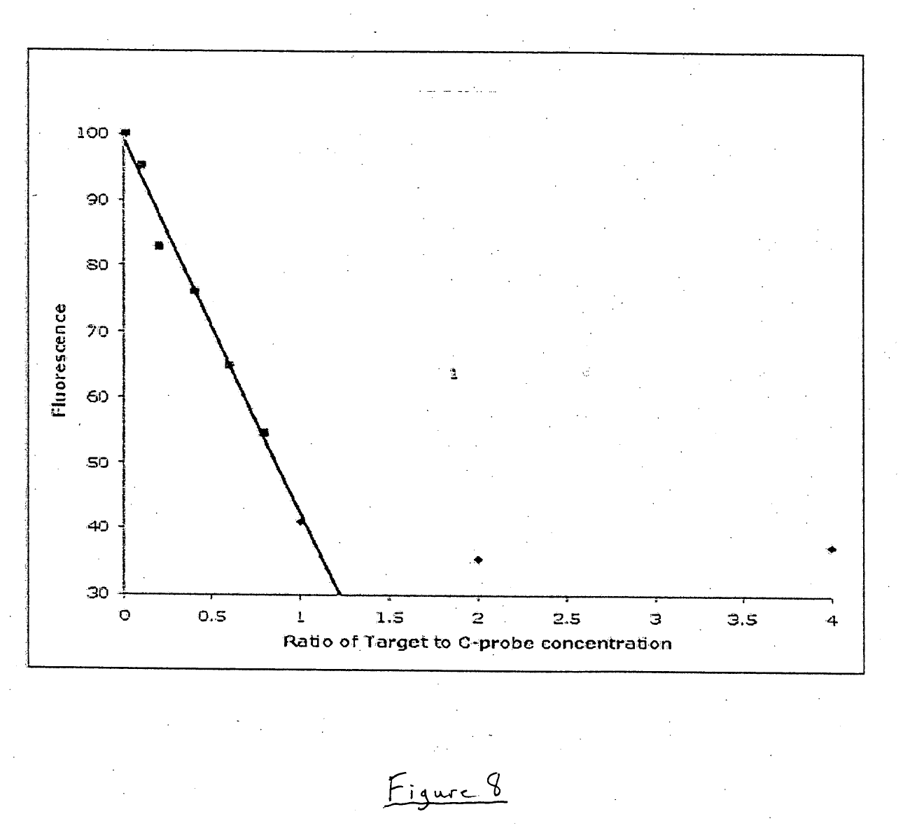

FIG. 9 is a hybridization curve demonstrating that the C-shaped probe of the present invention is not sensitive to excess target as compared with a binary pair control, wherein the fluorescence intensity of the controls decreased from 68% to 48% whereas the fluorescence of the C-probe failed to decrease appreciably beyond its existing intensity;

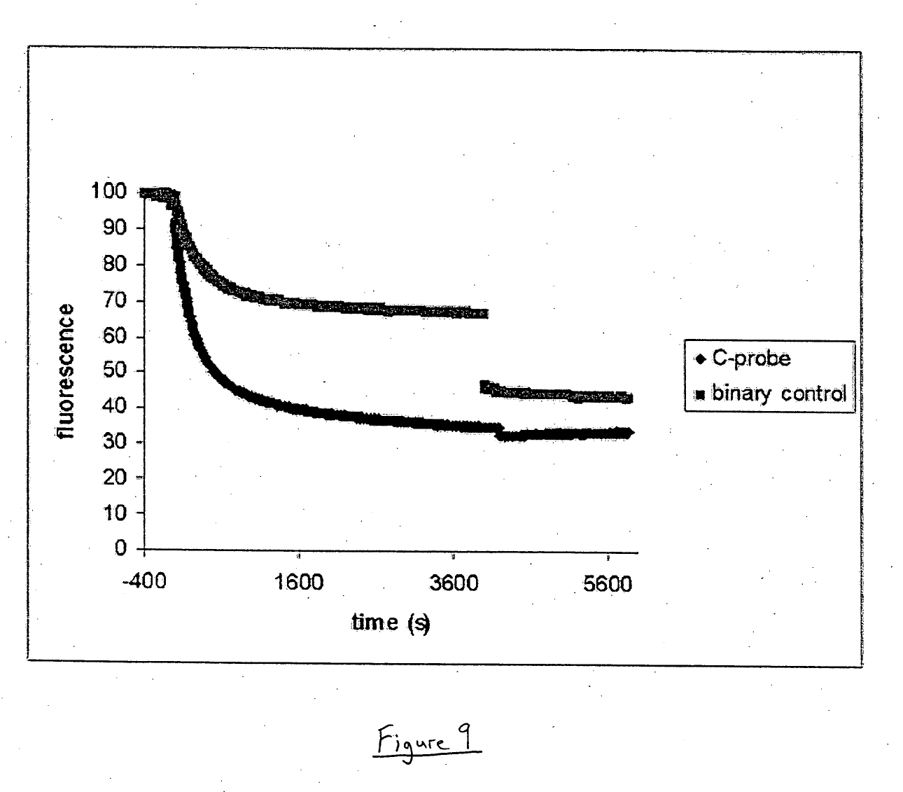

FIG. 10 is a chart demonstrating that the melting temperature of the C-shaped probe of the present invention increases with increasing arm length of the C-probe;

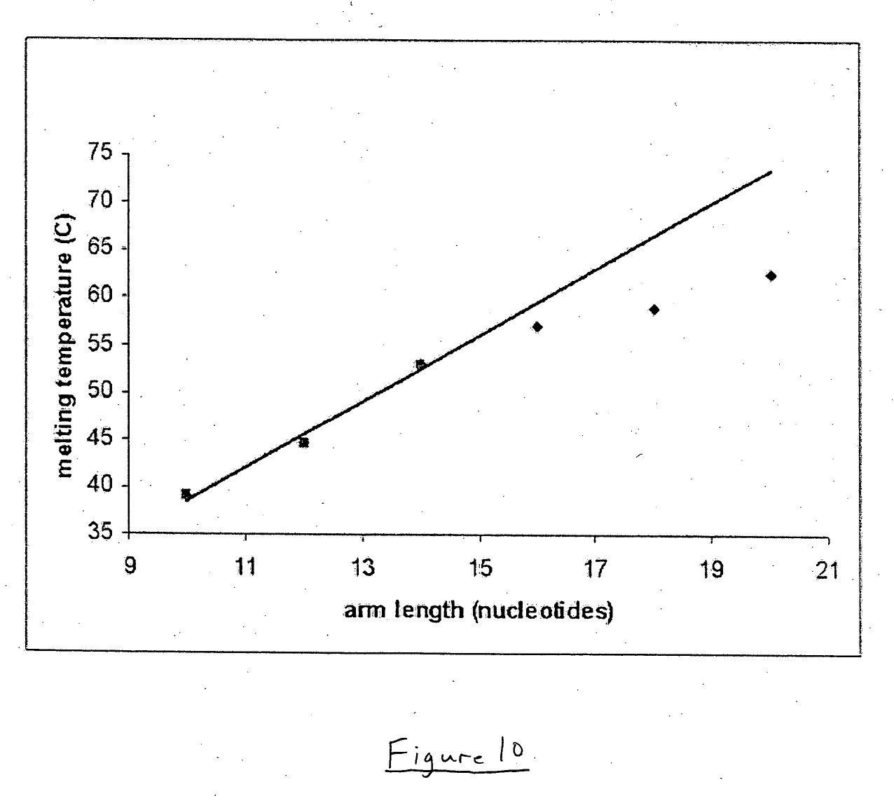

FIG. 11 is a bar chart demonstrating that the melting temperature higher for the C-shaped probe as compared to a binary pair control; and

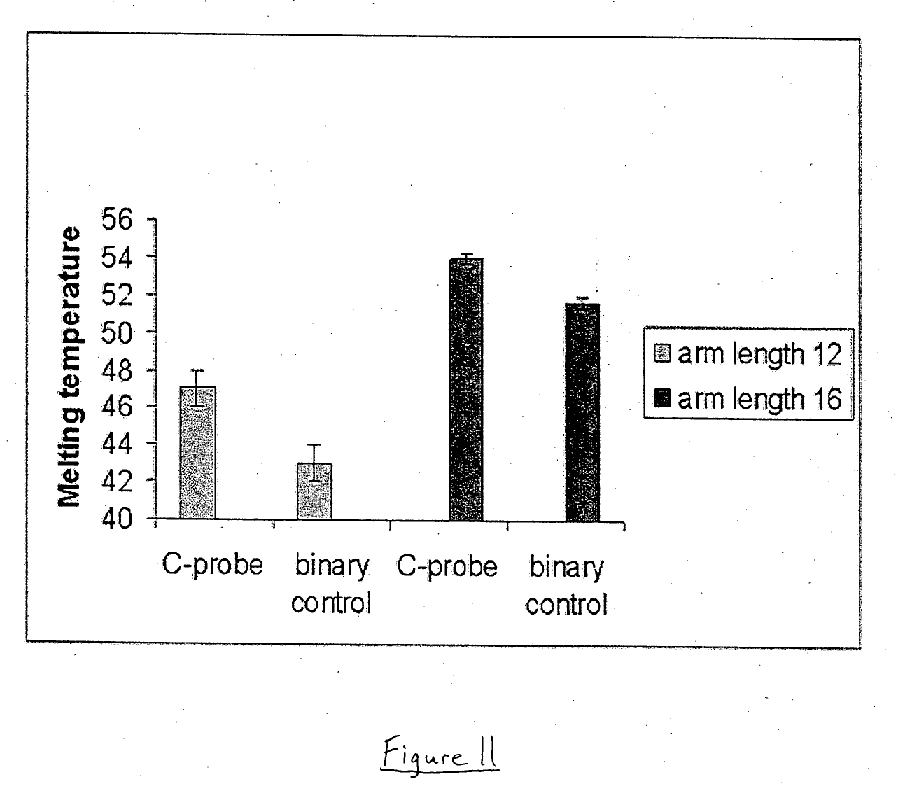

FIG. 12 illustrates the anchor effect of the C-shaped probe of the present invention.

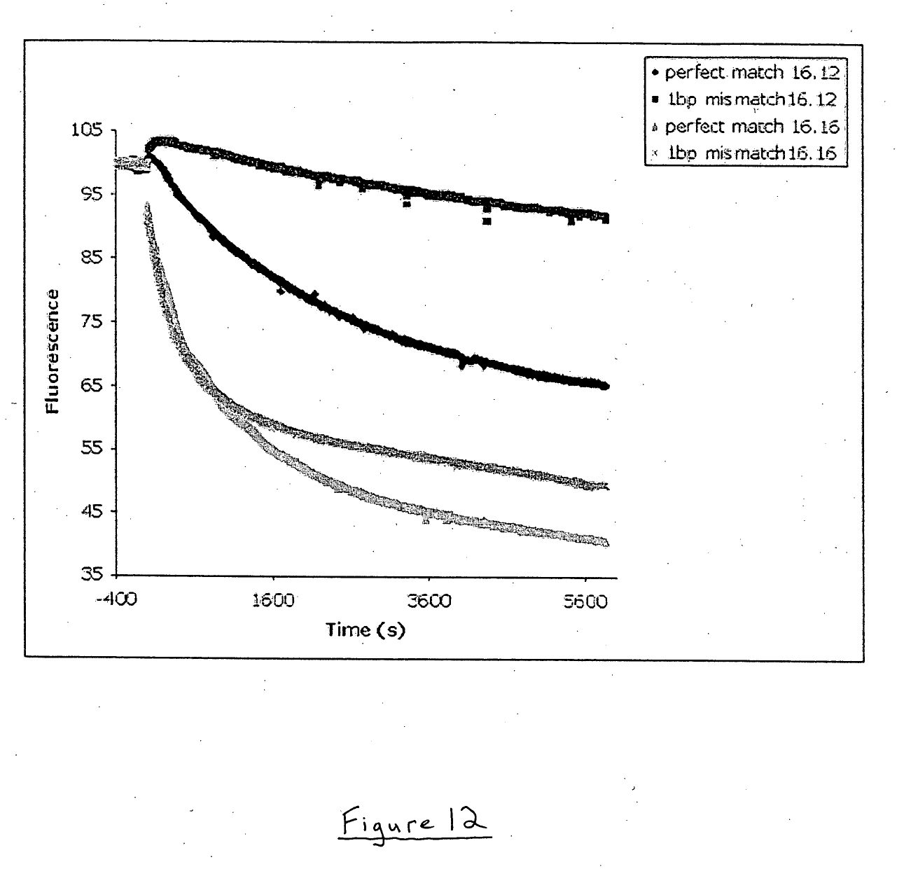

### DETAILED DESCRIPTION OF THE INVENTION

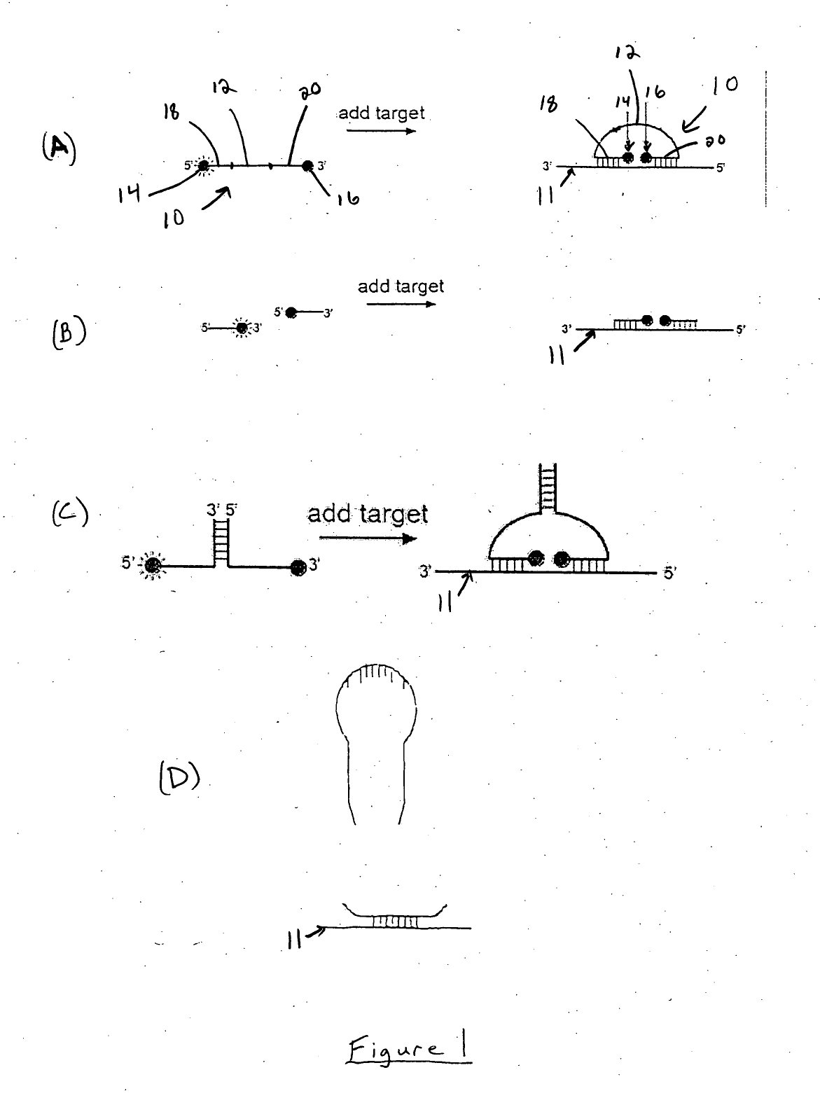

Generally, the present invention is a nucleic acid probe 10 used for the detection of a nucleic acid target sequence 11 (single or double stranded). The nucleic acid probe 10, which is preferably C-shaped when attached to a nucleic acid target sequence 11, utilizes two probes that are linked at the 5′ and 3′ ends of the probe 10 (See, FIG. 1A).

The probe 10 includes a linker structure 12 including a marker pair mechanism 14, 16. The linker structure 12 includes a 5′ marker mechanism 14 on the 5′ end of the probe 10 and a 3′ marker mechanism at the 3′ end of the probe 10. Preferably, the probe 10 includes a first nucleic acid arm 18 including any desired number of nucleic acids complementary to the target nucleic acid sequence 11, wherein the first nucleic acid arm 18 connects the 5′ marker mechanism 14 to the linker structure 12 at the 5′ end of the probe 10. Additionally, a second nucleic acid arm 20 including any desired number of nucleic acids complementary to the target nucleic acid sequence is included in the probe 10, wherein the second nucleic acid arm 20 connects the 3′ marker mechanism 16 to the linker structure 12 at the 3′ end of the probe 10. Both the first nucleic acid arm 18 and the second nucleic acid arm 20 include at least one nucleic acid. The length of the arms 18, 20 is determined by the number of nucleic acid molecules utilized in each arm. Each arm can be the same length or can vary from each other. The number of nucleic acid molecules in each arm 18, 20 varies according to design of the probe and/or the target nucleic acid sequence 11.

The term “marker pair mechanism” 14, 16 as used herein means two moieties that chemically and/or physically interact with each other. One moiety is identified as a 5′ marker mechanism 14, while the other moiety is identified as a 3′ marker mechanism 16. The marker pair mechanism 14, 16 can be a donor/acceptor pair, quencher/acceptor pair, and combinations thereof. The 5′ and 3′ marker mechanisms 14, 16 interact with each other wherein the 5′ marker mechanism 14 detectably alters at least one physically measurable characteristic of the 3′ marker mechanism 16 in a proximity-dependent manner. Alternatively, the 3′ marker mechanism 14 can detectably alter at least one physically measurable characteristic of the 5′ marker mechanism 16. Therefore, either the 5′ or 3′ marker mechanism 14, 16 can be the donor, quencher, or acceptor. Various marker pair mechanisms 14, 16 can be used with the present invention. Marker pair mechanisms 14, 16 include, but are not limited to, acceptor-donor protein pairs, protein-ligand pairs, enzyme-cofactor pairs, antibody-antigen pairs, protein-protein unit pairs, protein-protein subunit pairs, nucleic acid binding proteins-binding site pairs, luminescent-quenching label pairs, fluorophore-quencher label pairs (i.e., fluorescence resonance energy transfer (FRET) pairs, where the characteristic signal is fluorescence of a particular wavelength), and any other similar pairs known to those of skill in the art. Various luminescent labels can be used with the luminescent-quenching label pairs that include, but are not limited to, fluorescent labels, radioluminescent label, chemiluminescent label, bioluminescent label, and electrochemiluminescent label. The signal produced by the marker pair mechanisms 14, 16 can be the generation or reduction of a signal or label. Although one marker pair mechanism 14, 16 is used, multiple marker pair mechanisms can also be used with the present invention.

The term “5′ marker mechanism” 14 as used herein means, but is not limited to, a labeling moiety that can be detectable upon a chemical and/or physical interaction with the 3′ marker mechanism 16. The interaction occurs when the 5′ marker mechanism 14 is within close proximity of the 3′ marker mechanism 16. The 5′ marker mechanism is conjugated to 5′ end of the probe 10. Depending upon the marker pair mechanism being used, the 5′ marker mechanism can be a donor, acceptor, quencher, or other similar marker device known to those of skill in the art. Thus, the 5′ marker mechanism can create, generate, or dissipate a detectable identification signal.

The term “3′ marker mechanism” 16 as used herein means, but is not limited to, a labeling moiety that can be detectable upon a chemical and/or physical interaction with the 5′ marker mechanism 14. The interaction occurs when the 3′ marker mechanism 16 is within close proximity of the 5′ marker mechanism 14. The 3′ marker mechanism 16 is conjugated to the 3′ end of the probe 10 depending upon the design of the probe 10 and the location of the 5′ marker mechanism 14. Depending upon the marker pair mechanism 14, 16 being used, the 3′ marker mechanism 16 can be a donor, acceptor, quencher, or other similar marker device known to those of skill in the art. Thus, the 3′ marker mechanism 16 can create, generate, or dissipate a detectable identification signal.

The terms “linker,” “linker sequence,” or “linker structure” 12 as used herein means a structure made of compounds including, but is not limited to, deoxyribonucleotides (DNA), ribonucleotides (RNA), modified nucleotides, a carbon backbone, modified internucleotide linkages, or any other appropriate linking compounds known to those of skill in the art.

The term “nucleic acid” as used herein means a biological molecule or acid (ribonucleic acid or deoxyribonucleic acid) including adenine, guanine, cytosine, thymine, and uracil. The nucleic acid molecules or nucleotides are joined by covalent bonds (i.e., phosphodiester linkage).

The present invention has numerous advantages over the prior art. For example, the present invention has increased sensitivity. Sensitivity is the ability to correctly identify the presence or absence of target nucleic acids. After one of the arms 18, 20 of the probe 10 of the present invention binds to the target nucleic acids, the other arm immediately binds. If one of the arms 18, 20 becomes detached from the target nucleic acids, then it can quickly reattach because the sequence is held nearby to its target sequence by the linker structure 12. There is less loss of entropy with the probe 10 of the present invention because when the probe 10 binds, the reaction goes from two molecules to one complex. In contrast, there is a greater loss of entropy with the probes of the prior art because the reaction typically goes from three molecules (separate, unlinked probes) to one complex. Therefore, the probe of the present invention binds more tightly to the target nucleic acids.

The probe 10 of the present invention also hybridizes faster than probes of the prior art. After one of the arms 18, 20 of the probe binds, the other complementary arm is automatically nearby to its complementary target nucleic acid sequence because the arm is held nearby by the linker structure 12. As a result, the binding is quick and the binding of the two arms 18, 20 of the probe 10 is cooperative. The probe 10 of the present invention also would require fewer probes themselves in solution. Due to the increased strength of binding of the probe 10, the amount of probes needed is reduced. Since there is a less need for many probes, background noise is decreased.

An additional advantage of the probe 10 of the present invention is that it can hybridize with a wider range of target sequences. For example, melting temperatures for two neighboring sequences do not need to be at the same temperature. With the prior art probes, if one of the complementary sequences has more G-C pairs and therefore a different melting temperature, then the two probes may not bind to the target nucleic acids in equal ratios. As a result, decreased detection ability occurs. With the probe 10 of the present invention, the first 18 and second arms 20 of the probe 10 do not need to have the same melting temperatures and can be of different lengths of nucleotides and still effectively detect the target nucleic acids. If one arm of the probe 10 strongly binds, then the other arm receives some benefit and also strongly binds because it is held nearby with the linker structure 12.

A further advantage of the probe 10 of the present invention is that any length of a target nucleic acid sequence can be detected. In fact, with the probe 10 of the present invention, the target sequence does not have to be as long and/or contain as many nucleotides as is required with other prior art probes. Because there is stronger binding of the target to the probe 10 of the present invention, the first 18 and second arms 20 of the probe 10 that are complementary to the target nucleic acids can be any number of nucleotides. Further, the probe 10 of the present invention hybridizes better with double stranded DNA than existing molecular probes. The probe 10 has an enhanced signal, faster hybridization kinetics, and higher melting temperature than probes of the prior art.

Specifically comparing the probe 10 of the present invention with the binary probes of the prior art, the probe 10 of the present invention produces an identification signal that remains unchanged in the presence of excess target nucleic acids, while binary probes produce a decreased signal in the presence of excess target nucleic acids. Further, by using one arm of the probe 10 as an anchor, the other arm can vary in length to achieve maximum specificity for discriminating one nucleotide base-pair mismatches or to achieve maximum sensitivity so that the probe 10 can ignore one base-pair mismatches. Additionally, the present invention involves an intramolecular-like reaction or a type of chelation effect. As soon as one arm binds, the other arm is located nearby and binds quickly after. It is a type of cooperative binding.

The present invention is based on the general principles of fluorescence resonance energy transfer (FRET). When the first and second arms 18, 20 of the probe 10 of the present invention hybridize to the target sequence of nucleic acids, the marker mechanisms 14, 16 are in close proximity so that excitation or reduction of excitation of the marker pair mechanism occurs. For example, in one preferred embodiment, the 5′ marker mechanism 14 is a donor marker mechanism, which can create FRET to the 3′ marker mechanism 16, wherein the 3′ marker mechanism 16 is an acceptor marker mechanism. Detection by a spectrofluorometer or other similar device known to those of skill in the art can occur. If the target sequence is absent, however, then the probes are randomly distributed in the solution and the emission from the acceptor marker mechanism is equal to baseline. In the absence of the target nucleic acid sequence, no emission above baseline is detected from the acceptor marker mechanism upon excitation of the donor marker mechanism because the two sequences are too distant for effective FRET. In the presence of the target nucleic acid sequence, the probe 10 of the present invention forms a loop and hybridizes to the target presence of the target nucleic acids (See, FIG. 1A). The donor and acceptor marker mechanisms are in close proximity so that upon excitation of the donor marker mechanism, there is FRET and photons are detected from the acceptor marker mechanism.

The present invention has numerous embodiments. Generally, the present invention can be depicted with the following formula:
5′ end Xₙ-Aₙ-Zₙ-Bₙ—Y_{n 3}′ end
wherein,
X=5′ marker mechanism for producing an identification signal;
Y=3′ marker mechanism for producing an identification signal, wherein the identification signal is produced when the 3′ marker mechanism is in close physical proximity to the 5′ marker mechanism;
A=first nucleic acid sequence including at least one nucleic acid including, but not limited to, adenine, guanine, cytosine, thymine, and uracil;
B=second nucleic acid sequence including at least one nucleic acid including, but not limited to, adenine, guanine, cytosine, thymine, and uracil;
Z=linking segment made of compounds including, but not limited to, deoxyribonucleotides (DNA), ribonucleotides (RNA), modified nucleotides, a carbon backbone, modified internucleotide linkages, combinations thereof, and any other similar compounds known to those of skill in the art;
n=an integer≧1 indicating the number of compounds; and
dotted lines represent bonds (e.g., covalent bonds or phosphodiester linkage).

In one embodiment of the present invention, there is provided a nucleic acid probe 10 for identifying a target nucleic acid sequence. The nucleic acid probe 10 includes a linker structure 12 including a 5′ end and a 3′ end. The linker structure 12 is made of compounds including, but not limited to, deoxyribonucleotides (DNA), ribonucleotides (RNA), modified nucleotides, a carbon backbone, modified internucleotide linkages, combinations thereof, and any other similar linking compounds known to those of skill in the art.

The nucleic acid probe 10 also includes a 5′ marker mechanism 14 for producing an identifying signal a target nucleic acid, wherein the 5′ marker mechanism is conjugated to the 5′ end of the linker structure 12. The 5′ marker mechanism 14 is conjugated to the 5′ end of the linker structure 12 through a first nucleic acid sequence or arm 18. The first nucleic acid sequence or arm 18 is sequence specific for hybridizing with the targeted nucleic acid sequence. Further, the first nucleic acid sequence or arm 18 includes any number of nucleic acids.

The nucleic acid probe 10 further includes a 3′ marker mechanism 16 for producing an identifying signal a target nucleic acid sequence, wherein the 3′ marker mechanism 16 is conjugated to the 3′ end of the linker structure 12 and wherein identification of the target nucleic acid sequence occurs when the 5′ marker mechanism 14 and 3′ marker mechanism 16 are in close physical proximity to each other. The 3′ marker mechanism 16 is conjugated to the 3′ end of the linker structure 12 through a second nucleic acid sequence or arm 20. The second nucleic acid sequence or arm 20 is sequence specific for hybridizing with the targeted nucleic acid sequence. Further, the second nucleic acid sequence or arm 20 includes any number of nucleic acids.

The identification or identifying signal produced by the 5′ and 3′ marker mechanisms 14, 16 can be any signal including, but not limited to, acceptor-donor signals, protein-ligand signals, enzyme-cofactor signals, antibody-antigen signals, protein-protein unit signals, protein-protein subunit signals, nucleic acid binding proteins-binding site signals, luminescent-quenching signals, fluorophore-quencher label signals (fluorescence resonance energy transfer (FRET)), fluorescent signals, radioluminescent signals, chemiluminescent signals, bioluminescent signals, electrochemiluminescent signals, combinations thereof, and any other similar signals known to those of skill in the art. The identification or identifying signal can be the production, generation, creation, or reduction of any of the above-listed signals.

Another embodiment of the present invention is directed towards a nucleic acid probe 10′ for identifying a target nucleic acid sequence including a marker pair mechanism 14′, 16′ for producing an identification or identifying signal to identify a target nucleic acid sequence. Further included is a linker structure 12′ for linking marker pair mechanism 14′, 16′ together. As set forth above, the linker structure 12′ can be made of various material or compounds. Additionally, the nucleic acid probe 10′ includes a first and a second nucleic acid sequence 18′, 20′ for linking the marker pair mechanism 14′, 16′ to the linker structure 12′, wherein the first and second nucleic acid sequences 18′, 20′ hybridize with a target nucleic acid sequence. The length (i.e., the number of nucleic acids) of the first and second nucleic acid sequences 18′, 20′ can vary on the desired target nucleic acid sequence.

The marker pair mechanism 14′, 16′ can be any pair of moieties as set forth above. For example, the marker pair mechanism 14′, 16′ can be any pair of moieties including, but not limited to, acceptor-donor protein pairs, protein-ligand pairs, enzyme-cofactor pairs, antibody-antigen pairs, protein-protein unit pairs, protein-protein subunit pairs, nucleic acid binding proteins-binding site pairs, luminescent-quenching label pairs, fluorophore-quencher label pairs (fluorescence resonance energy transfer (FRET)) pairs, combinations thereof, and any other similar moiety pairs known to those of skill in the art. Further, the marker pair mechanism 14′, 16′ produces an identification signal by generating or reducing a signal including, but not limited to, a fluorescent signal, radioluminescent signal, chemiluminescent signal, bioluminescent signal, electrochemiluminescent signal, combinations thereof, and any other similar identification signals known those of skill in the art.

Another embodiment of the present invention is directed towards a biosensor. For example, if a fluorophore quencher pair 14″, 16″ is used on the two ends of the nucleic acid probe 10″ instead of a donor/acceptor pair, then this can function as a detector that turns dark when a certain threshold of nucleotides is detected. This is useful as biosensors for detecting the presence of infectious agents. The biosensor includes a fluorophore quencher pair 14″, 16″; and a linker sequence 12″ for linking the fluorophore quencher pair 14″, 16″. The linker sequence 12″ is made of compounds including, but not limited to, deoxyribonucleotides (DNA), ribonucleotides (RNA), modified nucleotides, a carbon backbone, modified internucleotide linkages, combinations thereof, and any other similar linking compounds known to those of skill in the art. Further, the fluorophore quencher pair 14″, 16″ is linked to the linker sequence 12″ through a first and second nucleic acid sequence 18″, 20″, respectively. These first and second nucleic acid sequences 18″, 20″ hybridize with the targeted nucleic acid sequence and vary in nucleic acid length. The first and second nucleic sequence 18″, 20″ can be any number of nucleic acids, depending upon the target nucleic acid sequence.

Other embodiments of the present invention are directed towards a kit including any of the above-described probes. The kit includes all required reagents and solutions, as is well known to those of skill in the art, needed to accomplish detection of the targeted nucleic acid sequence.

The probe 10 of the present invention can be made through numerous methods known to those of skill in the art. In one method, the nucleic acid sequence of the probe 10 is synthesized in an oligonucleotide-synthesizing machine. Then, a commercially available fluorophore is coupled to one end of the oligonucleotide. After purification via HPLC, the other fluorophore is coupled to the other end of the oligonucleotide. After a second round of purification via HPLC, the probe is ready for use. The reaction covalently links an EDANS moiety to the 5′ sulfhydryl group of the oligodeoxyribonucleotides that already had DABCYL coupled to the 3′ end.

The probe 10 of the present invention has numerous uses. The probe 10 can be used for imaging RNA or DNA within a cell. While molecular beacons as those set forth in the prior art can open up nonspecifically in certain areas of the cell, the probe 10 of the present invention does not give a false-positive signal. The probe 10 of the present invention can also be combined with multiple probes with each probe having a different colored fluorophore. Just as multiple molecular beacons can be used to detect different targets as part of the same assay, multiple probes can be mixed within the same assay to detect more than one target sequence. The probe 10 of the present invention can also be used to monitor real time PCR. Just as the molecular beacons of the prior art are used to monitor the progress of real time PCR reactions, the probe can do the same. Thus, the probe 10 of the present invention can be used for detecting a polymerase chain reaction product in a sample including the step of applying the probe 10 of the present invention to the PCR solution.

Since the probe of the present invention has a higher sensitivity than the molecular beacon of the prior art, earlier detection of the target nucleic acids with fewer cycles of the PCR occurs. The probe 10 of the present invention can also be used in a homogenous solution whereas some of the prior art require additional steps and washes. These improvements in probe design ultimately leads to better detection of bacteria in clinical specimens, monitoring of RNA in living cells, and detection of genetic mutations in the human genome.

The probe 10 of the present invention also is advantageous in monitoring RNA in a living cell. By attaching two molecular beacons with a linker as with the probe 10 of the present invention leads to advantages such as not having a decreased signal if there is excess target, enhancing the specificity, increased hybridization temperatures and increasing the FRET interaction.

The above discussion provides a factual basis for the use of the present invention described herein. The methods used with a utility of the present invention can be shown by the following non-limiting examples and accompanying figures.

### EXAMPLES

Materials and Methods for Examples:

General Methods in Molecular Biology:

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et. al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences (Testoni et al, 1996, Blood 87:3822.)

### Example One

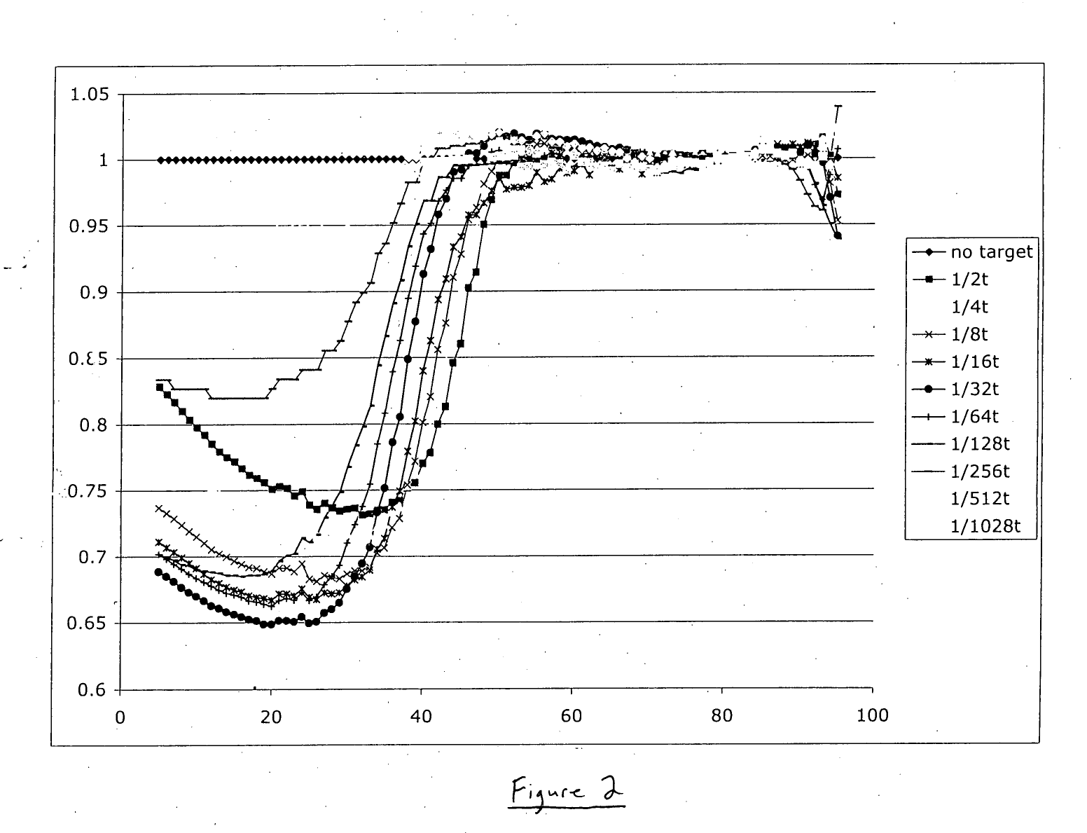

As set forth in FIG. 2, a hybridization curve of the probe and binary control demonstrates that the probe generates a stronger signal and hybridizes faster than the binary controls. A solution of the probe or the binary control with a 4-fold excess of the 3′ quencher control was maintained at 45° C. for 400 seconds. Then, a concentration matched perfectly complementary target was added. Upon addition of the target, the fluorescence of the probe decreased by 60% while the fluorescence of the binary control decreased by 40%. This proves that the hybridization kinetics is faster for the probe than the controls.

### Example Two

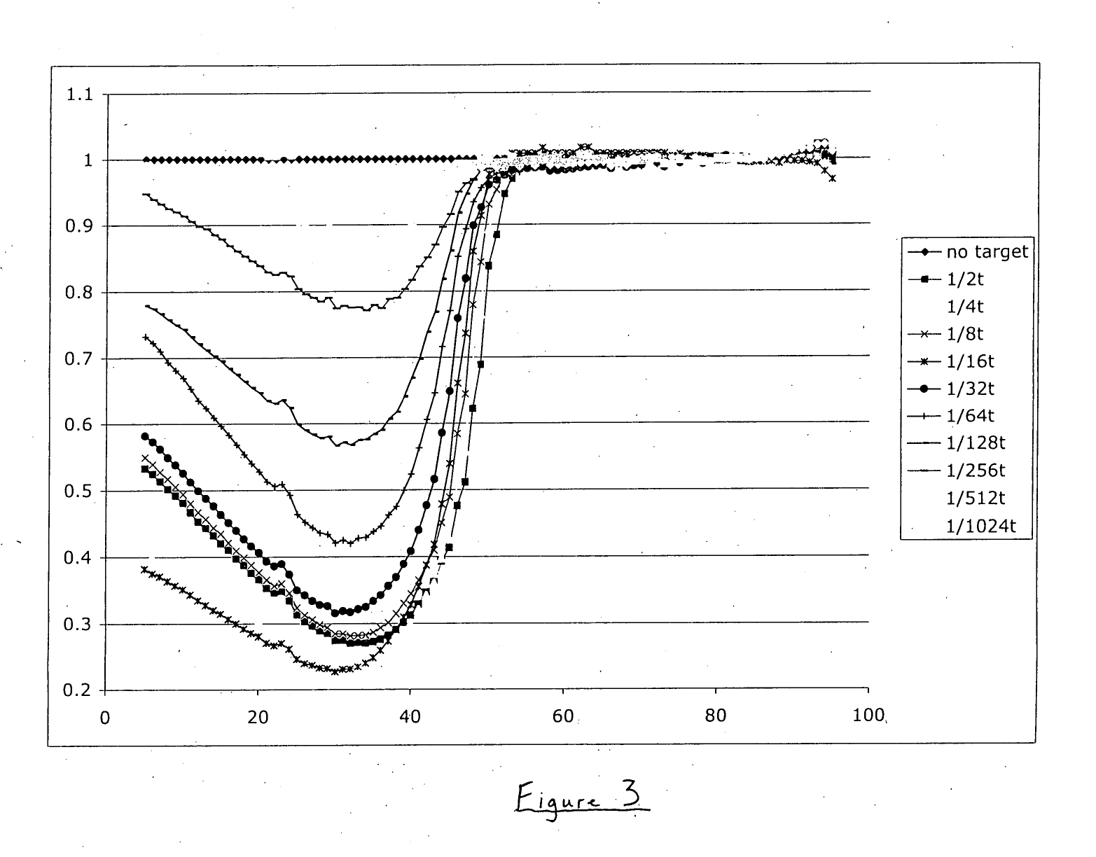

As set forth in FIG. 3, it is demonstrated that the C-shaped probe can quantitatively measure the concentration of a targeted nucleic acid sequence. Nine aliquots of the C-shaped probe were prepared with nine different concentrations of perfectly complementary targeted nucleic acid sequences. The ratio of target to probe concentration was 0.01, 0.1, 0.2, 0.4, 0.5, 0.8, 1, 2, and 4. In the range of excess probe, the signal varies approximately linearly with target concentration. Excess target does not appreciably decrease the signal of the probe. Therefore, the probe can be used to quantitatively determine the concentration of complementary target in solution.

### Example Three

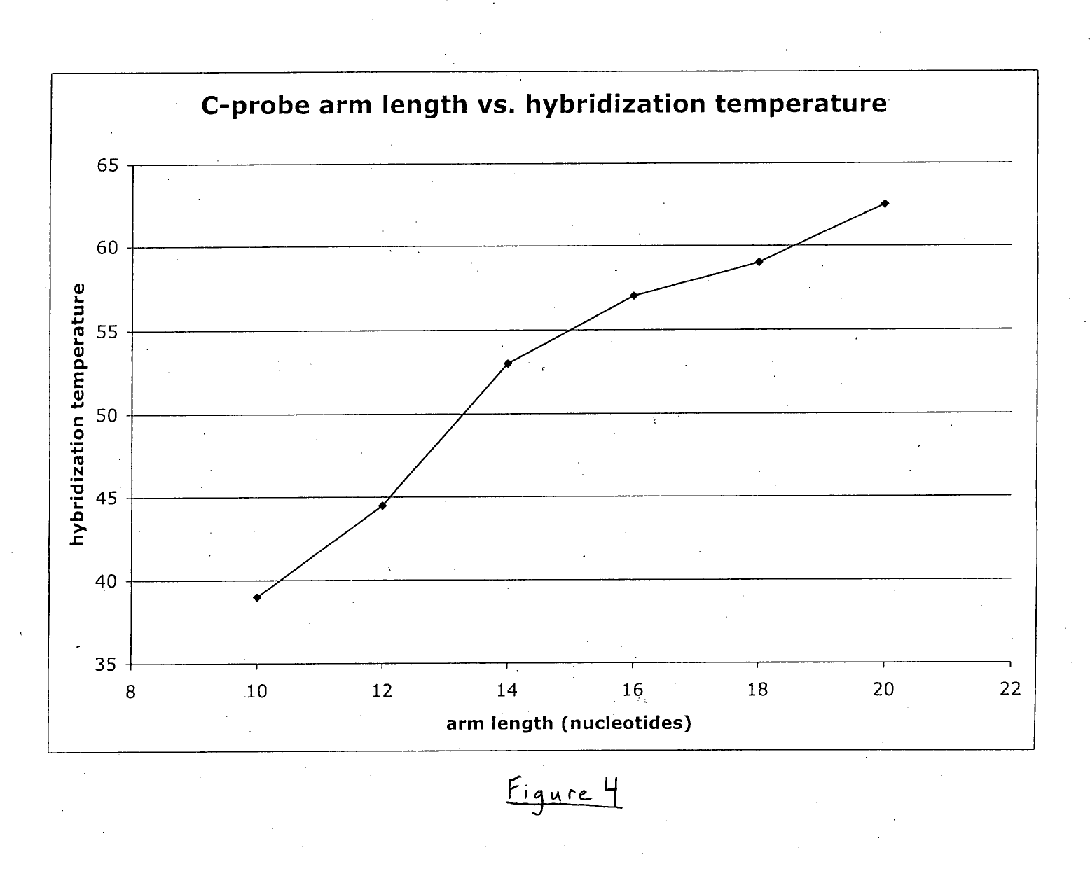

According to FIG. 4, the hybridization curve of the probe and binary control demonstrates that the probe is not sensitive to excess target. Solutions of the probe or the two control probes were maintained at 45° C. for 400 seconds. Then, at time 0 seconds, a 4-fold excess of perfectly complementary target was added. After 4100 seconds, a 200-fold excess of 3′ control quencher probe was added to both solutions. The fluorescence intensity of the controls decreased from 68% to 48% whereas the fluorescence of the probe failed to decrease appreciably beyond its existing intensity. This demonstrates that the two-control probes have a decreased signal when there is excess target, whereas the C-shaped probe does not lose signal when there is excess target.

### Example Four

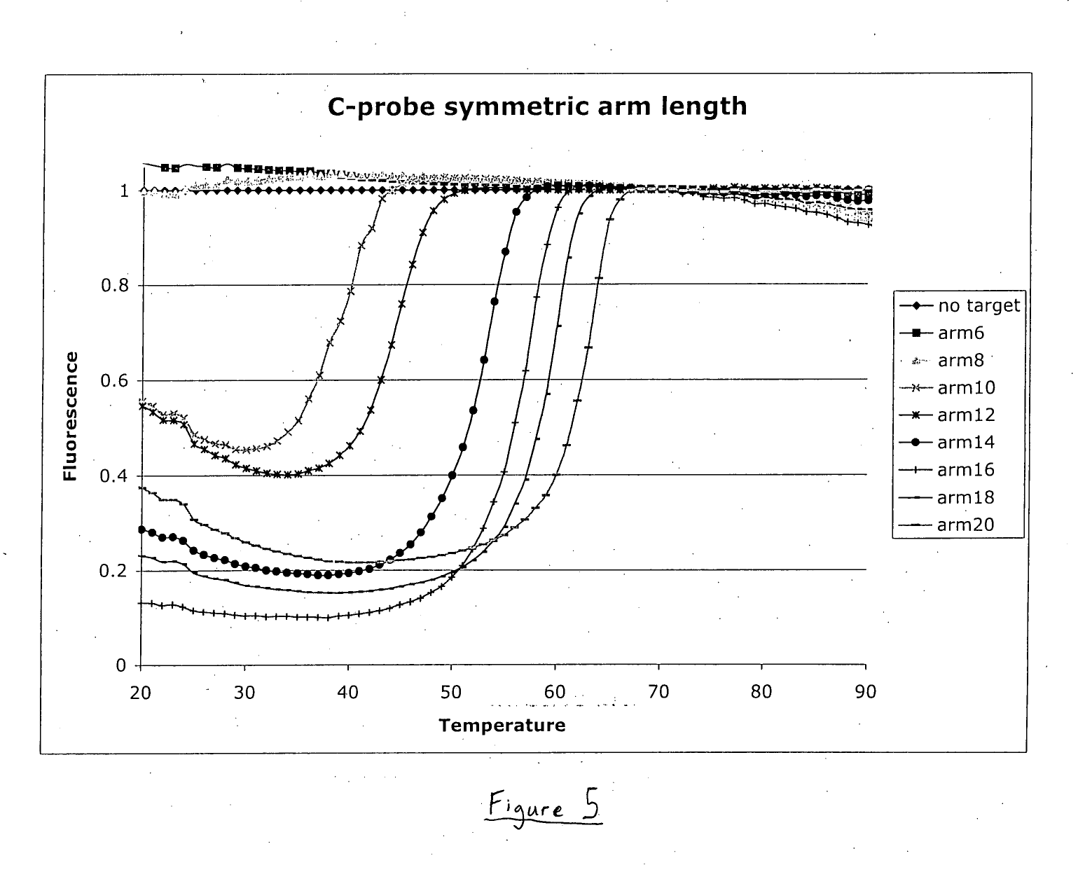

FIG. 5 illustrates that the melting temperature increases with increasing arm length of the probe. As the arm length of the probe increases from 10 to 20, the melting temperature increases. Due to linker strain for the higher arm lengths of 16, 18, and 20 nts., the melting temperature is lower than would otherwise be expected. The linear regression includes arm lengths of 10, 12, and 14 nts.

### Example Five

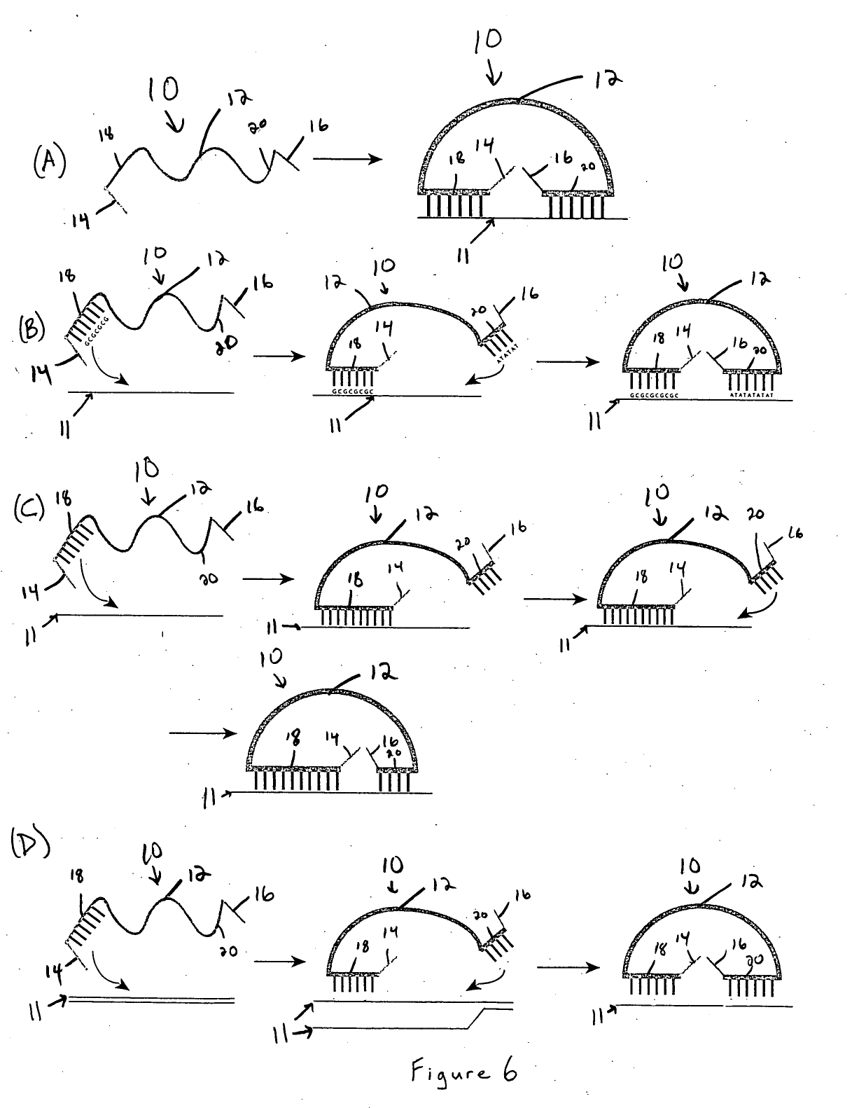

FIG. 6 demonstrates that the melting temperature is higher for the C-shaped probe than for a binary pair control. The melting temperature is the temperature at which there is a 50% change in fluorescent signal.
G=H−TS
T=(H−G)/S

The probe has a smaller AS than the binary control because there is a smaller loss in entropy (2 molecules to 1) as compared to the binary control (3 molecules to 1) upon hybridization. There is linker strain in the 16-armed probe when bound to the target, which increases the AS. For the 12-armed probe (arms span 24 nts., linker of 40 nts.) there is less loss of entropy upon hybridization due to less linker strain as compared to the 16-armed probe (arms span 32 nts., linker of 32 nts). Therefore, there is more of a difference in AS between the 12-armed probe and 12-armed binary control than the 16-armed probe and 16-armed binary control. The larger AS corresponds to a larger difference in melting temperatures. So, the 12-armed probe of the present invention has a higher melting temperature than the 12-armed binary control. The difference between the melting temperature of the 12-armed probe and the 12-armed binary control (about 5° C.) is greater than the difference between the melting temperature of the 16-armed probe and the 16-armed binary control (about 2° C.).

### Example Six

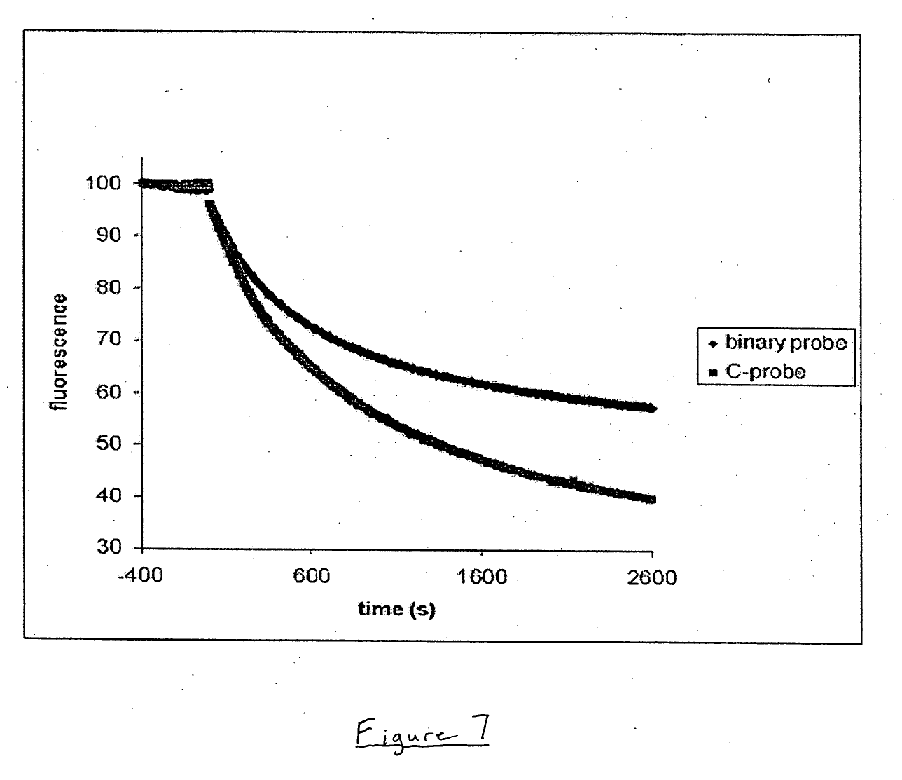

FIG. 7 demonstrates the anchor effect of the present invention. Solutions of the probe were maintained at 40° C. for 400 seconds. Then, either a molar-matched perfectly complementary target or a target containing a single nucleotide mismatch was added. The probe that had arm lengths of 16 and 12 nts. was not able to appreciably discriminate the mismatched target from the perfectly complementary target. The probe that had one arm length of 16 (the anchor arm) and the other arm of length 12 (the specificity arm) was able to discriminate between the perfectly complementary target and the target with the one nucleotide mismatch. The chelating ability of the probe with long arm lengths allows a probe that will not be allele discriminatory. Whereas the probe with a long arm as an anchor and another short specificity arm can effectively discriminate one base pair mismatches. Also, increasing the strain of the hybridized probe with a shorter linker increases the specificity of the probe.

The probe is an effective alternate to the binary probe system that is currently in widespread use. Specifically, the C-shaped probe has an enhanced signal, a higher melting temperature, and does not appreciably decrease its signal as does the binary probes to excess target. The C-shaped probe's insensitivity to excess target is useful in cases when a false negative signal is not wanted. This would be desired in early detection of biological agents.

By using one arm as a sensitive anchor to bind to the target, the other arm of the C-shaped probe can be made short to be extremely discriminatory of a perfectly complementary target or long to ignore single base pair mismatches. Furthermore, the ability to discriminate a one base pair mismatch can be further enhanced by adding a linker strain to the probe. While molecular beacons have been shown to be more specific for discriminating mismatches than the binary probes, requiring lower temperatures than the binary probes to hybridize to a one base pair mismatch, the C-shaped probe has the ability to be so specific that the probe cannot hybridize at any temperature to the target if the probe arm is short enough and with sufficient linker strain. The ability to make a super-specific probe is a major advancement over other probe designs.

The melting temperature of the probe is not significantly decreased by having a short second arm as it would for the binary probe where the melting temperature is solely determined by the shorter of the two probes. The fundamental design of coupling both arms of the probe via a linker allows cooperative interactions when binding to the target. Therefore, the melting temperature of the C-shaped probe is not determined by the shorter of the two arms, but by a combination of the melting temperature of the anchor arm, the melting temperature of the probing arm, and the linker strain.

### Example Seven

FIG. 2 is a plot of the fluorescence of the control lightcycler probes as a function of temperature in a series of different concentrations of target DNA present. FIG. 3 is a plot of the fluorescence of the probe as a function of temperature in a series of different concentrations of target DNA present. As the temperature goes down, hybridization of the probe to target occurs, and the fluorescent donor is fixed into a conformation immediately next to a fluorescent quencher on the 3′ end of the probe. From comparison of these two graphs, there are at least three main advantages of the probe of the present invention over the probes of the prior art (i.e., control lightcycler probes).

First, the probe achieves a maximum of 80% quenching whereas the control lightcycler probes only achieve a maximum of 35% quenching upon hybridization to a target sequence. Second, hybridization occurs at higher temperatures for the probe indicating that the probe has an increased sensitivity compared to the lightcycler probes of the prior art. Third, excess target does not decrease the fluorescent signal of the probe as it does to the control probe. When there is excess target, the 5′ lightcycler probe hybridizes to one target DNA and the 3′ lightcycler probe binds to a different target DNA leading to no signal detected. Both arms of the probe bind to the same target DNA even in the presence of excess target.

With regard to FIG. 4, this is a plot that shows that as the probe arm length is increased, hybridization of the probe to the target DNA increases at increasingly higher temperatures. For monitoring products in PCR, it is necessary to detect the target at temperatures above 50° C. FIG. 5 is a plot that shows as the probe arm length is increased to a length of 14 or 16, an increased fluorescence signal (as well as hybridization at higher temperatures) occurs. Arm 6 represents a probe arm length of 6 nucleotides as part of each arm.

Throughout this application, author and year and patents by number reference various publications, including United States patents. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used, is intended to be in the nature of words of description rather than of limitation.

Obviously, many modifications and variations of the present invention are possible in light of the above teachings. It is, therefore, to be understood that within the scope of the described invention, the invention may be practiced otherwise than as specifically described.

### REFERENCES

Backman A, Lantz P G, Radstrom P, Olcen P “Evaluation of an extended diagnostic PCR assay for detection and verification of the common causes of bacterial meningitis in CSF and other biological samples” Molecular and Cellular Probes, 13 (1): 49-60 February 1999.
Cox C J, Kempsell K E, Gaston J SH “Investigation of infectious agents associated with arthritis by reverse transcription PCR of bacterial rRNA” Arthritis Research & Therapy, 5 (1): R1-R8 2003.
Greisen, K., Loeffelholz, M., Purohit, A., Leong, D. “PCR Primers and Probes for the 16S Ribosomal-RNA Gene of Most Species of Pathogenic Bacteria, including Bacteria Found in Cerebrospinal-Fluid,” Journal of Clinical Microbiology, 32 (2): 335-351 February 1994.
Jalava J, Skurnik M, Toivanen A, Toivanen P, Eerola E “Bacterial PCR in the diagnosis of joint infection” Annals of the Rheumatic Diseases, 60 (3): 287-289 March 2001.
Jaton, K., Sahli, R., Bille, J. “Development of Polymerase Chain Reaction Assays for Detection of LISTERIA-MONOCYTOGENES in Clinical Cerebrospinal-fluid Samples,” Journal of Clinical Microbiology, (8): 1931-1936 August 1992
Kristiansen, Be., Ask, E., Jenkins, A., Fermer, C., Radstrom, P., and Skold, O., “Rapid Diagnosis of Miningococcal Mennigitis by Polymerase Chain Reaction,” Lancet, 337 (8757) 1568-1569 (Jun. 29, 1991).
Schuurman T, de Boer R F, Kooistra-Smid A M D, van Zwet M “Prospective study of use of PCR amplification and sequencing of 16S ribosomal DNA from cerebrospinal fluid for diagnosis of bacterial meningitis in a clinical setting” Journal of Clinical Microbiology, 42 (2): 734-740 February 2004.
Stuhimeier R, Broell H, Stuhlmeier K M “Fast and sensitive method for the detection of bacteria in synovial fluid by multiplex PCR” Arthritis Research & Therapy, 5: 84 Suppl. 1 2003.
Tyagi, S., and FR Kramer, Nature Biotechnology, Vol. 14, p. 303 (March 1996).
van Haeften R, Palladino S, Kay I, Keil T, Heath C, Waterer G W “A quantitative LightCycler PCR to detect Streptococcus pneumoniae in blood and CSF” Diagnostic Microbiology and Infectious Disease, 47 (2): 407-414 October 2003
Wellinghausen N, Wirths B, Franz A R, Karolyi L, Marre R, Reischl U “Algorithm for the identification of bacterial pathogens in positive blood cultures by real-time LightCycler polymerase chain reaction (PCR) with sequence-specific probes” Diagnostic Microbiology and Infectious Disease, 48 (4): 229-241 April 2004.

What is claimed is:

1. A nucleic acid probe for identifying a target nucleic acid sequence comprising:
a linker structure including a 5′ end and a 3′ end;
5′ marker means for producing an identifying signal to a target nucleic acid sequence, wherein said 5′ marker means is conjugated to said 5′ end of said linker structure; and
3′ marker means for producing an identifying signal to the target nucleic acid sequence, wherein said 3′ marker means is conjugated to said 3′ end of said linker structure and wherein identification of the target nucleic acid sequence occurs when said 5′ marker means and 3′ marker means are in close physical proximity to each other.

2. The nucleic acid probe according to claim 1, wherein said linker structure is made of material selected from the group consisting of deoxyribonucleotides (DNA), ribonucleotides (RNA), modified nucleotides, a carbon backbone, modified internucleotide linkages, and combinations thereof.

3. The nucleic acid probe according to claim 1, wherein said 5′ marker means includes a first nucleic acid sequence for linking said 5′ marker means to said 5′ end of said linker structure and for hybridizing with a target nucleic acid sequence.

4. The nucleic acid probe according to claim 1, wherein said 3′ marker means includes a second nucleic acid sequence for linking said 3′ marker means to said 3′ end of said linker structure and for hybridizing with a target nucleic acid sequence.

5. The nucleic acid probe according to claim 1, wherein said identifying signal is selected from the group consisting of acceptor-donor signals, protein-ligand signals, enzyme-cofactor signals, antibody-antigen signals, protein-protein unit signals, protein-protein subunit signals, nucleic acid binding proteins-binding site signals, luminescent-quenching signals, fluorophore-quencher label signals (fluorescence resonance energy transfer (FRET)), fluorescent signals, radioluminescent signals, chemiluminescent signals, bioluminescent signals, electrochemiluminescent signals, and combinations thereof.

6. A nucleic acid probe for identifying a target nucleic acid sequence comprising marker pair means for producing an identification signal to identify a target nucleic acid sequence; and linker means for linking said marker pair means together.

7. The nucleic acid probe according to claim 6, wherein said linker means is made of compounds selected from the group consisting of deoxyribonucleotides (DNA), ribonucleotides (RNA), modified nucleotides, a carbon backbone, modified internucleotide linkages, and combinations thereof.

8. The nucleic acid probe according to claim 6, wherein said marker pair means includes a first and a second nucleic acid sequence for linking said marker pair means to said linker means, wherein said first and said second nucleic acid sequences hybridize with a target nucleic acid sequence.

9. The nucleic acid probe according to claim 6, wherein said marker pair means is selected from the group consisting of acceptor-donor protein pairs, protein-ligand pairs, enzyme-cofactor pairs, antibody-antigen pairs, protein-protein unit pairs, protein-protein subunit pairs, nucleic acid binding proteins-binding site pairs, luminescent-quenching label pairs, fluorophore-quencher label pairs (fluorescence resonance energy transfer (FRET)), and combinations thereof.

10. The nucleic acid probe according to claim 6, wherein said identification signal is the generation of a signal selected from the group consisting of a fluorescent signal, radioluminescent signal, chemiluminescent signal, bioluminescent signal, and electrochemiluminescent signal.

11. The nucleic acid probe according to claim 6, wherein said identification signal is the reduction of a signal selected from the group consisting of a fluorescent signal, radioluminescent signal, chemiluminescent signal, bioluminescent signal, and electrochemiluminescent signal.

12. A biosensor comprising a fluorophore quencher pair; and a linker sequence for linking said fluorophore quencher pair.

13. The biosensor according to claim 12, wherein said linker sequence is made of material selected from the group consisting of deoxyribonucleotides (DNA), ribonucleotides (RNA), modified nucleotides, a carbon backbone, modified internucleotide linkages, and combinations thereof.

14. The biosensor according to claim 12, wherein said fluorophore quencher pair includes a first and a second nucleic acid sequence for linking said fluorophore quencher pair to said linker sequence, wherein said first and said second nucleic acid sequences hybridize with a target nucleic acid sequence.

15. A nucleic acid sequence probe for identifying a nucleic acid target sequence, wherein said nucleic acid sequence probe has the formula:
5′ end Xₙ-Aₙ-Zₙ-Bₙ—Y_{n 3}′ end
wherein,
X=5′ marker means for producing an identification signal;
Y=3′ marker means for producing an identification signal, wherein said identification signal is produced when said 3′ marker means is in close physical proximity to said 5′ marker means;
A=first nucleic acid sequence including at least one nucleic acid selected from the group consisting of adenine, guanine, cytosine, thymine, and uracil;
B=second nucleic acid sequence including at least one nucleic acid selected from the group consisting of adenine, guanine, cytosine, thymine, and uracil;
Z=linking segment made of compounds selected from the group consisting of deoxyribonucleotides (DNA), ribonucleotides (RNA), modified nucleotides, a carbon backbone, modified internucleotide linkages, and combinations thereof;
n=an integer≧1 and indicates the number of compounds; and
dotted lines represent bonds.

16. A method of detecting a polymerase chain reaction (PCR) product in a sample by applying the probe according to claim 1 to a polymerase chain reaction (PCR) solution.

17. A kit for detecting a target nucleic acid sequence comprising the nucleic acid probe of claim 1.
